SEQUENCE LISTING (6 sequences)

SEQ ID NO: 1 - protein, 10 aa, homo Sapiens
CVNNDETCEQ

SEQ ID NO: 2 - DNA, 30 bp, Homo Sapiens
tgcgttaataatgatgaaacctgtgagcag

SEQ ID NO: 3 - protein, 15 aa, Homo Sapiens
QDIEASHYRGYGNSD

SEQ ID NO: 4 - DNA, 45 bp, Homo Sapiens
caggatattgaagcatcccactacagaggctacggaaactctgat

SEQ ID NO: 5 - protein, 9 aa, Homo Sapiens
DLKDDQKEM

SEQ ID NO: 6 - DNA, 26 bp, Homo Sapiens
acttaaaagatgatcaaaaagaaatg

(19) United States
(12) Patent Application Publication
(10) Pub. No.: US 2010/0166748 A1
(43) Pub. Date: Jul. 1, 2010

(54) C5 Antigens and Uses Thereof

(75) Inventors: Braydon Charles Guild, Cambridge, MA (US); Mark Taylor Keating, Cambridge, MA (US); Mariusz Milik, Cambridge, MA (US); Dmitri Mikhailov, Cambridge, MA (US); Michael Roguska, Cambridge, MA (US); Igor Splawski, Cambridge, MA (US); Kehao Zhao, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/532,261

(22) PCT Filed: Mar. 19, 2008
(86) PCT No.: PCT/EP2008/053321

§ 371 Date: Sep. 21, 2009

Related U.S. Application Data
(60) Provisional application No. 60/896,408, filed on Mar. 22, 2007.

(51) Int. Cl. A61K 39/395; A61K 38/16; C07K 7/08; C07K 7/06; C07H 21/04; A61P 27/02; C12N 15/63; C12N 5/10; C12P 21/06

(52) U.S. Cl. 424/133.1; 424/139.1; 514/12; 530/326; 530/328; 536/23.1; 435/320.1; 435/325; 435/69.1

(57) ABSTRACT

The present invention pertains to the use of a complement inhibitor in methods of treatment of ocular disorders and the use of a complement inhibitor in the manufacture of a medicament in the treatment of an ocular disorder.

### BACKGROUND OF THE INVENTION

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. Pathogenesis of new choroidal vessel formation is poorly understood, but factors such as inflammation, ischemia, and local production of angiogenic factors are thought to be important.

The genes for the complement system proteins have been determined to be strongly associated with a person's risk for developing macular degeneration. The complement system is a crucial component of the innate immunity against microbial infection and comprises a group of proteins that are normally present in the serum in an inactive state. These proteins are organized in three activation pathways: the classical, the lectin, and the alternative pathways. Molecules on the surface of microbes can activate these pathways resulting in the formation of protease complexes known as C3-convertases. The classical pathway is a calcium/magnesium-dependent cascade, which is normally activated by the formation of antigen-antibody complexes. It can also be activated in an antibody-independent manner by the binding of C-reactive protein complexed with ligand and by many pathogens including gram-negative bacteria. The alternative pathway is a magnesium-dependent cascade which is activated by deposition and activation of C3 on certain susceptible surfaces (e.g. cell wall polysaccharides of yeast and bacteria, and certain biopolymer materials).

The alternative pathway participates in the amplification of the activity of the classical pathway and the lectin pathway. Activation of the complement pathway generates biologically active fragments of complement proteins, e.g. C3a, C4a and C5a anaphylatoxins and C5b-9 membrane attack complexes (MAC), which mediate inflammatory responses through involvement of leukocyte chemotaxis, activation of macrophages, neutrophils, platelets, mast cells and endothelial cells, increased vascular permeability, cytolysis, and tissue injury.

Complement component C5 is the major component of the final pathway common to the lectin, classical and alternative pathways in the complement cascade. The cleavage of C5 by the C5 convertases of the alternative and classical pathways yields C5b and C5a fragments. Both C5a and C5b are proinflammatory molecules. C5a is a powerful anaphylotoxin. C5a binds the C5a receptor (C5aR) and stimulates the synthesis and release from human leukocytes of proinflammatory cytokines such as TNF-α, IL-1β, IL-6 and IL-8. C5b serves as the nucleation site for the assembly of C5b-9 (C5b, C6, C7, C8 and C9) also as known as the terminal complement complex or the membrane attack complex (MAC) that penetrates cell membranes forming a pore, which at sublytic concentrations can contribute to proinflammatory cell activation while at lytic concentrations it leads to cell death. Reducing the formation of C5b-9 (MAC) and the generation of C5a may be required for the inhibition of inflammatory responses contributing to AMD. Inhibiting the cleavage of C5 that is catalyzed by the C5 convertases of the alternative and classical pathways may be critical to the therapeutic treatment of AMD.

Despite current treatment options for treating diseases and disorders associated with the classical or alternative component pathways, particularly AMD, there remains a need for finding specific targets that lead to treatments which are effective and well-tolerated.

### SUMMARY OF THE INVENTION

The present invention relates to C5 proteins, including sequences selected from the group consisting of SEQ ID 1-6, fragments thereof and methods of making or using said proteins. The present invention also relates to vectors and recombinant host cells comprising C5 polynucleotides and polypeptides. Another aspect of the invention is to provide methods for identifying test agents that modulate C5 complement component activity and for identifying binding partners of C5 antigens. Utility of the isolated C5 proteins of the present invention is based on the discovery of specific epitopes of C5 that are involved in biological activities associated with dysregulation of complement activity, specifically, macular degeneration.

The present invention provides the use of C5 proteins or fragments thereof as immunogens to generate binding molecules that bind to at least one epitope of C5 selected from the group consisting of SEQ ID 1-6, for preventing, treating and/or delaying diseases or disorders involving dysregulation of complement pathway activity

In other aspects, the invention provides binding molecules which inhibit at least one component of the alternate complement pathway, and encompass methods of making or using said binding molecules for preventing, treating and/or delaying ocular diseases or disorders, such as AMD.

In certain other aspects, the invention provides a method of treating or preventing ocular diseases or disorders, or delaying its progression, the method comprising administering an effective amount of antibodies which specifically bind to one or more epitopes of C5 to thereby inhibit C5 protein function in the complement pathway systems of a subject in need of such treatment.

In another aspect of the invention, a pharmaceutical composition for use in the therapeutic or prophylactic methods of treatment is provided, which composition comprises a protein inhibitor of complement C5 function, a protein inhibitor of binding of C5b to C6 or a pharmaceutically acceptable salt thereof, together with one or more pharmaceutically acceptable diluents or carriers therefore.

The invention further provides use of binding molecules capable of inhibiting the alternate complement pathway in the manufacture of a medicament for the treatment of an ocular disease or disorder, or for delaying their progression, which protein is capable of inhibiting C5 protein function or production of the MAC complex.

The invention also provides methods of identifying a C5 epitope or nucleic acid encoding the same in a sample by contacting the sample with a binding molecule that specifically binds to the epitope or nucleic acid encoding such polypeptide, e.g. an antibody, and detecting complex formation, if present. Also provided are methods of identifying a compound or binding molecule that modulates the activity of C5 proteins by contacting C5 epitopes with such compound and determining whether the C5 protein activity is modified.

In yet another aspect, the invention provides a method of determining the presence of or predisposition in a subject a disorder associated with complement pathway dysregulation, comprising the steps of providing a sample from the subject and measuring the amount of C5 protein in the subject sample. The amount of the particular protein or inhibition in the subject sample is then compared to the amount of that protein or inhibition in a control sample. A control sample is preferably taken from a matched individual, i.e., an individual of similar age, sex, or other general condition but who is not suspected of having complement pathway-associated conditions. Alternatively, the control sample may be taken from the subject at a time when the subject is not suspected of having conditions associated with complement pathway dysregulation. In some aspects, the compound or binding molecule of interest is detected using a binding molecule, specifically an antibody, as described herein.

In a further aspect of the invention, a screening method is provided for binding C5 proteins in a serum sample comprising the step of allowing competitive binding between antibodies in a sample and a known amount of antibody (anti-C5) of the invention or a functionally equivalent variant or fragments thereof and measuring the amount of the known antibodies.

In another aspect, the present invention relates to a diagnostic kit for detecting disorders associated with complement pathway dysregulation, comprising compounds or binding molecules of the invention and a carrier in suitable packaging. The kit preferably contains instructions for using an antibody to detect the presence of a C5 epitope. Preferably the carrier is pharmaceutically acceptable.

### DESCRIPTION AND PREFERRED EMBODIMENTS

As used herein “compounds” or “compounds of the present invention” shall mean proteins including peptides, oligonucleotides, peptidomimetcs, homologues, analogues and modified or derived forms thereof. The compounds of the invention preferably include nucleic acid sequences, fragments and derivatives thereof selected from the group consisting of SEQ ID Nos 2, 4 and 6. The invention also includes mutant or variant sequences, any of whose bases may be changed from the corresponding SEQ ID Nos 2, 4 and 6 while still encoding a protein, preferably an antigenic protein selected from the group consisting of SEQ ID Nos 1, 3 and 5.

“Binding molecules” shall mean antibodies, organic molecules, proteins including peptides, oligonucleotides, peptidomimetics, homologues, analogues and modified or derived forms thereof which bind to the compounds of the invention, preferably compounds selected from SEQ ID Nos 1-6.

Derivatives or analogs of the compounds and binding molecules of the invention include, but are not limited to, molecules comprising regions that are substantially homologous to the nucleic acids or proteins disclosed herein, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions (Ausubel et al., 1987).

The present invention provides antigenic epitopes of C5 protein, binding molecules which specifically bind to linear or nonlinear epitopes, methods of making and using such antigenic epitopes and binding molecules. The inventors are the first to describe epitopes of C5 having the sequences selected from SEQ ID Nos 1-6 which can be modulated for preventing, treating or ameliorating disorders associated with complement pathway dysregulation, preferably ocular diseases and disorders.

Certain ocular diseases and disorders which can be treated or prevented by the present invention comprise inflammation and/or neovascularization of at least a portion of the eye. Certain, non-limiting diseases and disorders can be used to treat or prevent by the methods provided herein include macular degeneration, diabetic ocular diseases and disorders, ocular edema, ischemic retinopathy, optic neuritis, cystoid macular edema, retinal diseases and disorders, pathologic myopia, retinopathy of prematurity, vascularized, rejecting, or otherwise inflamed corneas (with or without corneal surgery or transplantation), keratoconjunctivitis sicca or dry eye. In certain aspects, preferred ocular diseases and disorders suitable for treatment or prevention by the compounds, binding molecules and methods of the invention include those selected from age-related macular degeneration, diabetic retinopathy, diabetic macular edema, and retinopathy of prematurity. Other ocular diseases potentially amenable to such a therapeutic approach include internal and external ocular inflammatory disorders such as uveitis, scleritis, episcleritis, conjunctivitis, keratitis, orbital cellulitis, ocular myositis, thyroid orbitopathy, lacrimal gland or eyelid inflammation.

“Ocular diseases or disorders” as defined in this application comprise, but are not limited to, diabetic ocular diseases or disorders, ocular edema, ischemic retinopathy with neovascularization, optic neuritis, cystoid macular edema (CME), retinal disease or disorder such as neovascular pathologic myopia, retinopathy of prematurity (ROP), vascularized, rejecting, or otherwise inflammed corneas (with or without corneal surgery or transplantation), keratoconjunctivitis sicca or dry eye. Other ocular diseases potentially amenable to such a therapeutic approach include internal and external ocular inflammatory disorders such as uveitis, scleritis, episcleritis, conjunctivitis, keratitis, orbital cellulitis, ocular myositis, thyroid orbitopathy, lacrimal gland or eyelid inflammation.

“Diabetic ocular diseases or disorders” as defined in this application comprises, but is not limited to diabetic retinopathy (DR), diabetic macular edema (DME), proliferative diabetic retinopathy (PDR).

Particular antigenic epitopes of the invention are encoded by SEQ ID Nos 1 to 6 and complements thereof.

Particular antigenic epitopes have an amino acid sequence at least 85%, preferably 90%, more preferably 95% identical to SEQ ID 1, 3 and 6.

Three surface exposed antigenic epitopes are identified on C5 proteins. The epitopes are based on three linear amino acid sequences, two on the alpha chain and one on the beta chain of complement component C5, as antigenic sites for binding including:

1). The amino acid sequence comprising CVNNDETCEQ (SEQ ID No. 1) on C5 alpha chain, encoded by nucleotide sequence TGCGTTAATAATGATGAAACCTGTGAGCAG (SEQ ID NO. 2); 2). The amino acid sequence comprising QDIEASHYRGYGNSD (SEQ ID No 3) on C5 alpha chain, encoded by nucleotide sequence CAGGATATTGAAGCATCCCACTACAGAGGCTACGGAAACTCTGAT (SEQ ID No. 4); 3). The amino acid sequence comprising DLKDDQKEM (SEQ ID No 5) on C5 beta chain, encoded by nucleotide sequence ACTTAAAAGATGATCAAAAAGAAATG (SEQ ID No. 6).

### Polynucleotides and Polypeptides

Isolated polypeptides and polynucleotides of the invention can be produced by any suitable method known in the art. Such methods range from direct protein synthetic methods to constructing a DNA sequence encoding isolated polypeptide sequences and expressing those sequences in a suitable transformed host.

Standard methods may be applied to synthesize an isolated polypeptide sequence of interest using standard methods of in vitro protein synthesis.

In one aspect of a recombinant method, a DNA sequence is constructed by isolating or synthesizing a DNA sequence encoding a wild type protein of interest. Optionally, the sequence may be mutagenized by site-specific mutagenesis to provide functional analogs thereof, or modified by any other means, e.g., by fusing to another gene sequence, thus generating fusion proteins, or by deleting specific parts of the gene sequence, resulting in the expression of a protein that lacks specific parts compared to the wild-type form. For example, a transmembrane domain can be deleted, thus creating a secreted version of a protein that in its original state is membrane anchored.

Another method of constructing a DNA sequence encoding a polypeptide of interest would be by chemical synthesis using an oligonucleotide synthesizer. Such oligonucleotides may be preferably designed based on the amino acid sequence of the desired polypeptide, and preferably selecting those codons that are favored in the host cell in which the recombinant polypeptide of interest will be produced. For example, a DNA oligomer containing a nucleotide sequence coding for the epitopes of SEQ ID Nos 1, 3 or 5 may be synthesized. In one feature, several small oligonucleotides coding for portions of these epitopes may be synthesized and then ligated. The individual oligonucleotides typically contain 5′ or 3′ overhangs for complementary assembly. A complete amino acid sequence may be used to construct a back-translated gene.

Once assembled (by synthesis, polymerase chain reaction, site-directed mutagenesis, or by any other method), the mutant DNA sequences encoding a particular isolated polypeptide of interest will be inserted into an expression vector and operatively linked to an expression control sequence appropriate for expression of the protein in a desired host. Proper assembly may be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host transformed by said vector.

The choice of expression control sequence and expression vector will depend upon the choice of the corresponding host. A wide variety of expression host/vector combinations may be employed. Useful expression vectors for eukaryotic hosts, include, for example, vectors comprising expression control sequences from SV40, bovine papilloma virus, retrovirus, adenovirus and cytomegalovirus. Useful expression vectors for bacterial hosts include known bacterial plasmids, such as plasmids from Escherichia coli, including pCR1, pBR322, pMB9 and their derivatives, wider host range plasmids, such as M13 and filamentous single-stranded DNA phages. Preferred E. coli vectors include pL vectors containing the lambda phage pL promoter (U.S. Pat. No. 4,874,702), pET vectors containing the T7 polymerase promoter and the pSP72 vector. Useful expression vectors for yeast cells, for example, include the 2 g and centromere plasmids.

Further, within each specific expression vector, various sites may be selected for insertion of these DNA sequences. These sites are usually designated by the restriction endonuclease which cuts them. They are well-recognized by those of skill in the art. It will be appreciated that a given expression vector useful in this invention need not have a restriction endonuclease site for insertion of the chosen DNA fragment. Instead, the vector may be joined by the fragment by alternate means.

The expression vector, and the site chosen for insertion of a selected DNA fragment and operative linking to an expression control sequence, is determined by a variety of factors such as: the number of sites susceptible to a particular restriction enzyme, the size of the polypeptide, how easily the polypeptide is proteolytically degraded, and the like. The choice of a vector and insertion site for a given DNA is determined by a balance of these factors.

To provide for adequate transcription of the recombinant constructs of the invention, a suitable promoter/enhancer sequence may preferably be incorporated into the recombinant vector, provided that the promoter/expression control sequence is capable of driving transcription of a nucleotide sequence encoding the polypeptide of interest. Any of a wide variety of expression control sequences may be used in these vectors. Such useful expression control sequences include the expression control sequences associated with structural genes of the foregoing expression vectors. Examples of useful expression control sequences include, for example, the-early and late promoters of SV40 or adenovirus, the lac system, the trp system, the TAC or TRC system, the major operator and promoter regions of phage lambda, for example pL, the control regions of fd coat protein, the promoter for 3-phosphoglycerate kinase or other glycolytic enzymes, the promoters of acid phosphatase, e.g., Pho5, the promoters of the yeast α-mating system and other sequences known to control the expression of genes of prokaryotic or eukaryotic cells and their viruses, and various combinations thereof. Many of the vectors mentioned are commercially available.

Any suitable host may be used to produce in quantity the isolated compounds of the invention, including bacteria, fungi (including yeasts), plants, insects, mammals, or other appropriate animal cells or cell lines, as well as transgenic animals or plants. More particularly, these hosts may include well known eukaryotic and prokaryotic hosts, such as strains of E. coli, Pseudomonas, Bacillus, Streptomyces, fungi, yeast (e.g., Hansenula), insect cells such as Spodoptera firugiperda (SF9), and HIGH FIVE, animal cells such as Chinese hamster ovary (CHO), mouse cells such as NS/0 cells, African green monkey cells, COS1, COS 7, BSC 1, BSC 40, and BMT 10, and human cells, as well as plant cells.

Promoters which may be used to control the expression of polypeptides in eukaryotic cells include, but are not limited to, the SV40 early promoter region, the promoter contained in the 3′ long terminal repeat of Rous sarcoma virus, the herpes thymidine kinase promoter, the regulatory sequences of the metallothionine gene.

In case the polypeptide is expressed in plants, plant expression vectors should be used comprising the nopaline synthetase promoter region or the cauliflower mosaic virus 35S RNA promoter and the promoter for the photosynthetic enzyme ribulose biphosphate-carboxylase.

In case the polypeptide is expressed in yeast or other fungi, promoter elements should be chosen such as the Gal 4 promoter, the ADC (alcohol dehydrogenase) promoter, PGK (phosphoglycerolkinase) promoter, alkaline phosphatase promoter.

In case the polypeptide is expressed in transgenic animals, the following animal transcriptional control regions can be used, which exhibit tissue specificity and have been utilized in transgenic animals: elastase I gene control region which is active in pancreatic cells; insulin gene enhancers for promoters which are active in pancreatic cells; immunoglobulin gene enhancers or promoters which are active in lymphoid cells; the cytomegalovirus early promoter and enhancer regions; mouse mammary tumor virus control region which is active in testicular, breast, lymphoid and mast cells; albumin gene control region which is active in liver; .alpha.-fetoprotein gene control region which is active in liver; α-antitrypsin gene control region which is active in the liver; β-globin gene control region which is active in myeloid cells, myelin basic protein gene control region which is active in oligodendrocyte cells in the brain; myosin light chain-2 gene control region which is active in skeletal muscle; and gonadotropic releasing hormone gene control region which is active in the hypothalamus.

Operative linking of a DNA sequence to an expression control sequence includes the provision of a translation start signal in the correct reading frame upstream of the DNA sequence. If the particular DNA sequence being expressed does not begin with a methionine, the start signal will result in an additional amino acid (methionine) being located at the N-terminus of the product. If a hydrophobic moiety is to be linked to the N-terminal methionyl-containing protein, the protein may be employed directly in the compositions of the invention. Yet, methods are available in the art to remove N-terminal methionines from polypeptides expressed with them. For example, certain hosts and fermentation conditions permit removal of substantially all of the N-terminal methionine in vivo.

It should be understood that not all vectors and expression control sequences will function equally well to express a given isolated polypeptide. Neither will all hosts function equally well with the same expression system. However, one of skill in the art may make a selection among these vectors, expression control systems and hosts without undue experimentation.

Successful incorporation of these polynucleotide constructs into a given expression vector may be identified by three general approaches: (a) DNA-DNA hybridization, (b) presence or absence of “marker” gene functions, and (c) expression of inserted sequences. In the first approach, the presence of the gene inserted in an expression vector can be detected by DNA-DNA hybridization using probes comprising sequences that are homologous to the inserted gene. In the second approach, the recombinant vector/host system can be identified and selected based upon the presence or absence of certain “marker” gene functions (e.g., thymidine kinase activity, resistance to antibiotics such as G418, transformation phenotype, occlusion body formation in baculovirus, etc.) caused by the insertion of foreign genes in the vector. For example, if the polynucleotide is inserted so as to interrupt a marker gene sequence of the vector, recombinants containing the insert can be identified by the absence of the marker gene function. In the third approach, recombinant expression vectors can be identified by assaying the foreign gene product expressed by the recombinant vector. Such assays can be based, for example, on the physical or functional properties of the gene product in bioassay systems.

Recombinant nucleic acid molecules which encode modified protein therapeutics may be obtained by any method known in the art (Maniatis et al., 1982, Molecular Cloning; A Laboratory Manual, Cold Spring Harbor Laboratory, Cold Spring Harbor, N.Y.) or obtained from publicly available clones. Modifications comprise but are not limited to deletions, insertions, point mutations, fusions to other polypeptides. In some embodiments of the invention, a recombinant vector system may be created to accommodate sequences encoding the therapeutic of interest in the correct reading frame with a synthetic hinge region. Additionally, it may be desirable to include, as part of the recombinant vector system, nucleic acids corresponding to the 3′ flanking region of an immunoglobulin gene including RNA cleavage/polyadenylation sites and downstream sequences. Furthermore, it may be desirable to engineer a signal sequence upstream of the modified protein therapeutic to facilitate the secretion of the protein therapeutic from a cell transformed with the recombinant vector. This is particularly of interest where a normally membrane-bound protein is modified in a way so that it will be secreted instead.

Proteins produced by a transformed host can be purified according to any suitable method. Such standard methods include chromatography (e.g., ion exchange, affinity, and sizing column chromatography), centrifugation, differential solubility, or by any other standard technique for protein purification. For immunoaffinity chromatography, the protein of interest may be isolated by binding it to an affinity column comprising antibodies that were raised against said protein or a cross-reactive protein and were affixed to a stationary support. to give a substantially pure protein. By the term “substantially pure” is intended that the protein is free of the impurities that are naturally associated therewith. Substantial purity may be evidenced by a single band by electrophoresis. Isolated proteins can also be characterized physically using such techniques as proteolysis, nuclear magnetic resonance, and X-ray crystallography.

### Antisense, Ribozyme, Triple Helix RNA Interference and Aptamer Techniques

Another aspect of the invention relates to the use of the compounds and/or modified compounds as therapeutics. In some aspect, nucleic acids are produced inside cells via means of gene transfer vectors. In other aspects, these nucleic acids are directly administered to the mammalian subject in vivo, including, for example, four different techniques described below: antisense, ribozyme, RNA interference and aptamers.

Antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Antisense

As used herein, “antisense” therapy refers to administration or in situ generation of oligonucleotide molecules or their derivatives which specifically hybridize (e.g., bind) under cellular conditions, with the cellular mRNA and/or genomic DNA encoding one or more epitopes of C5 so as to inhibit expression of or activation of C5, e.g., by inhibiting transcription and/or translation of C5 proteins. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy that relies on specific binding to oligonucleotide sequences.

An antisense construct of the present invention can be delivered, for example, as an expression plasmid which, when transcribed in the cell, produces RNA which is complementary to sequences of the cellular mRNA which encodes a C5 antigenic protein. Alternatively, the antisense construct is an oligonucleotide probe that is generated ex vivo and which, when introduced into the cell causes inhibition of expression by hybridizing with the mRNA and/or genomic sequences of a C5 polynucleotides. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). With respect to antisense DNA, oligodeoxyribonucleotides derived from sequences selected from SEQ ID 2, 4 or 6 are preferred.

Antisense approaches involve the design of oligonucleotides (either DNA or RNA) that are complementary to mRNA encoding epitopes of C5 protein. The antisense oligonucleotides will bind to the mRNA transcripts and prevent translation. Absolute complementarity, although preferred, is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. Sequences complementary to the 3′ untranslated sequences of mRNAs have also been shown to be effective at inhibiting translation of mRNAs. Therefore, oligonucleotides complementary to either the 5′ or 3′ untranslated, non-coding regions of a gene could be used in an antisense approach to inhibit translation of that mRNA. Oligonucleotides complementary to the 5′ untranslated region of the mRNA should include the complement of the AUG start codon. Antisense oligonucleotides complementary to mRNA coding regions are less efficient inhibitors of translation but could also be used in accordance with the invention. Whether designed to hybridize to the 5′, 3′ or coding region of mRNA, antisense nucleic acids should be at least six nucleotides in length, and are preferably less than about 100 and more preferably less than about 50, 25, 17 or 10 nucleotides in length.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, PCT Publication No. WO88/09810) or the blood-brain barrier (see, e.g., PCT Publication No. WO89/10134), hybridization-triggered cleavage agents or intercalating agents. To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; β-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further aspect, the antisense oligonucleotide is an anomeric oligonucleotide. An anomeric oligonucleotide forms specific double-stranded hybrids with complementary RNA in which, contrary to the usual units, the strands run parallel to each other. The oligonucleotide is a 2′-O-methylribonucleotide, or a chimeric RNA-DNA analogue.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the methods known in the art, methylphosphonate oligonucleotides can be prepared by use of controlled pore glass polymer supports

While antisense nucleotides complementary to the coding region of an mRNA sequence can be used, those complementary to the transcribed untranslated region and to the region comprising the initiating methionine are most preferred.

The antisense molecules can be delivered to cells that express C5 proteins in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigen expressed on the target cell surface) can be administered systematically.

However, it may be difficult to achieve intracellular concentrations of the antisense sufficient to suppress translation on endogenous mRNAs in certain instances. Therefore a preferred approach utilizes a recombinant DNA construct in which the antisense oligonucleotide is placed under the control of a strong pol m or pol II promoter. The use of such a construct to transfect target cells in the patient will result in the transcription of sufficient amounts of single stranded RNAs that will form complementary base pairs with the endogenous hedgehog signaling transcripts and thereby prevent translation. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region, the promoter contained in the 3′ long terminal repeat of Rous sarcoma virus, the herpes thymidine kinase promoter, the regulatory sequences of the metallothionein gene (Brinster et al, 1982, Nature 296:3942), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

### Ribozymes

Ribozyme molecules designed to catalytically cleave C5 mRNA transcripts can also be used to prevent translation of mRNA (See, e.g., PCT International Publication WO90/11364, published Oct. 4, 1990; U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been published in International patent application WO88/04300. The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and should be delivered to cells that express C5 proteins in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

### Triple Helix Formation

Alternatively, endogenous C5 gene expression can be reduced by targeting deoxyribonucleotide sequences complementary to the regulatory region of the gene (i.e., the promoter and/or enhancers) to form triple helical structures that prevent transcription of the gene in target cells in the body.

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

### RNA Interference

The discovery that RNA interference (RNAi) seems to be a ubiquitous mechanism to silence genes suggests an alternative, novel approach to decrease gene expression, which is able to overcome the limitations of the other approaches outlined above. Short interfering RNAs (siRNAs) are at the heart of RNAi. The antisense strand of the siRNA is used by an RNAi silencing complex to guide cleavage of complementary mRNA molecules, thus silencing expression of the corresponding gene.

The present invention—leveraging RNAi—thus differs from other nucleic acid based strategies (antisense and ribozyme methods) in both approach and effectiveness: (a) compared to antisense strategies, RNAi leverages a catalytic process, i.e., a small amount of siRNA is capable of decreasing the concentration of the target gene mRNA within the target cell. As antisense is based on a stoichiometric process, a much larger concentration of effector molecules is required within the target cell, i.e., a concentration is required that is equal to or greater than the concentration of endogenous mRNA. Thus, as RNAi is a catalytic process, a lower amount of effector molecules (i.e., siRNAs) is sufficient to mediate a therapeutic effect. (b) Compared to ribozymes (which have a catalytic function as well), RNAi seems to be a more flexible strategy, which allows targeting a higher variety of target sequences and thus offers more flexibility in construct design. Moreover, design of RNAi constructs is fast and convenient as the artisan can design those constructs based on the sequence information of the RNAi target gene. With ribozymes, more trial-and-error experiments and more sophisticated design algorithms are required as ribozymes are more complex in nature. Last, (c) RNAi is more efficacious in vivo compared to ribozymes as RNAi leverages ubiquitous, endogenous cell machinery.

The present invention also differs from protein-based strategies, as RNAi does not require the expression of non-endogenous proteins (such as artificial transcription factors), thus lowering the risk of an unintended immune response.

In summary, RNAi-mediated down-regulation of gene expression is a novel mechanism with clear advantages over existing gene expression down-regulation approaches.

RNAi constructs comprise double stranded RNA that can specifically block expression of a target gene. Accordingly, RNAi constructs can act as antagonists by specifically blocking expression of a particular gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs.

Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50.degree. C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs. Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, α-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer doublestranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end. These double-stranded siRNA structures can then be directly introduced to cells, either by passive uptake or a delivery system of choice.

In certain aspects, the siRNA constructs can be generated by processing of longer doublestranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred features, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one aspect, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

In other features, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects that may be caused by the sequence-independent dsRNA response. In such embodiments, the use of local delivery systems and/or agents which reduce the effects of interferon or PKR are preferred.

In certain aspects, the RNAi construct is in the form of a hairpin structure (named as hairpin RNA). The hairpin RNAs can be synthesized exogenously or can be formed by transcribing from RNA polymerase III promoters in vivo. Preferably, such hairpin RNAs are engineered in cells or in an animal to ensure continuous and stable suppression of a desired gene. It is known in the art that siRNAs can be produced by processing a hairpin RNA in the cell.

In yet other aspects, a plasmid is used to deliver the double-stranded RNA, e.g., as a transcriptional product. In such features, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain aspects, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Exemplary RNAi constructs that specifically recognize a particular gene, or a particular family of genes can be selected using methodology outlined in detail above with respect to the selection of antisense oligonucleotide. Similarly, methods of delivery RNAi constructs include the methods for delivery antisense oligonucleotides outlined in detail above. In general, it is anticipated that any of the foregoing methods that decrease the presence or translation of C5 proteins or activity.

The design of the RNAi expression cassette does not limit the scope of the invention. Different strategies to design an RNAi expression cassette can be applied, and RNAi expression cassettes based on different designs will be able to induce RNA interference in vivo. (Although the design of the RNAi expression cassette does not limit the scope of the invention, some RNAi expression cassette designs are included in the detailed description of this invention and below.)

Features common to all RNAi expression cassettes are that they comprise an RNA coding region which encodes an RNA molecule which is capable of inducing RNA interference either alone or in combination with another RNA molecule by forming a double-stranded RNA complex either intramolecularly or intermolecularly.

Different design principles can be used to achieve that same goal and are known to those of skill in the art. For example, the RNAi expression cassette may encode one or more RNA molecules. After or during RNA expression from the RNAi expression cassette, a double-stranded RNA complex may be formed by either a single, self-complementary RNA molecule or two complementary RNA molecules. Formation of the dsRNA complex may be initiated either inside or outside the nucleus.

The RNAi target gene does not limit the scope of this invention and may be any gene that participates in C5 activity or expression. Thus, the choice of the RNAi target gene is not limiting for the present invention: The artisan will know how to design an RNAi expression cassette to down-regulate the gene expression of any RNAi target gene of interest. Depending on the particular RNAi target gene and method of delivery, the procedure may provide partial or complete loss of function for the RNAi target gene.

### Aptamers

Aptamers are a non-naturally occurring nucleic acid having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. The target in case of the, present invention is a component of the Hedgehog signaling pathway.

Aptamers are identified based on the SELEX process (Gold, et al., PNAS 94:59-64, 1997). In its most basic form, the SELEX process may be defined by the following series of steps:

A candidate mixture of nucleic acids of differing sequence is prepared. The candidate mixture generally includes regions of fixed sequences (i.e., each of the members of the candidate mixture contains the same sequences in the same location) and regions of randomized sequences. The fixed sequence regions are selected either: (a) to assist in the amplification steps described below, (b) to mimic a sequence known to bind to the target, or (c) to enhance the concentration of a given structural arrangement of the nucleic acids in the candidate mixture. The randomized sequences can be totally randomized (i.e., the probability of finding a base at any position being one in four) or only partially randomized (e.g., the probability of finding a base at any location can be selected at any level between 0 and 100 percent).

The candidate mixture is contacted with the selected target under conditions favorable for binding between the target and members of the candidate mixture. Under these circumstances, the interaction between the target and the nucleic acids of the candidate mixture can be considered as forming nucleic acid-target pairs between the target and those nucleic acids having the strongest affinity for the target.

The nucleic acids with the highest affinity for the target are partitioned from those nucleic acids with lesser affinity to the target. Because only an extremely small number of sequences (and possibly only one molecule of nucleic acid) corresponding to the highest affinity nucleic acids exist in the candidate mixture, it is generally desirable to set the partitioning criteria so that a significant amount of the nucleic acids in the candidate mixture (approximately 5-50%) are retained during partitioning.

Those nucleic acids selected during partitioning as having the relatively higher affinity to the target are then amplified to create a new candidate mixture that is enriched in nucleic acids having a relatively higher affinity for the target.

By repeating the partitioning and amplifying steps above, the newly formed candidate mixture contains fewer and fewer weakly binding sequences, and the average degree of affinity of the nucleic acids to the target will generally increase. Taken to its extreme, the SELEX process will yield a candidate mixture containing one or a small number of unique nucleic acids representing those nucleic acids from the original candidate mixture having the highest affinity to the target molecule.

In order to produce nucleic acids desirable for use as a pharmaceutical, it is preferred that the nucleic acid ligand (1) binds to the target in a manner capable of achieving the desired effect on the target; (2) be as small as possible to obtain the desired effect; (3) be as stable as possible; and (4) be a specific ligand to the chosen target. In most situations, it is preferred that the nucleic acid ligand have the highest possible affinity to the target.

The SELEX patent applications describe and elaborate on this process in great detail. Included are targets that can be used in the process; methods for partitioning nucleic acids within a candidate mixture; and methods for amplifying partitioned nucleic acids to generate enriched candidate mixture. The SELEX patent applications also describe ligands obtained to a number of target species, including both protein targets where the protein is and is not a nucleic acid binding protein. The SELEX method further encompasses combining selected nucleic acid ligands with lipophilic or non-immunogenic, high molecular weight compounds in a diagnostic or therapeutic complex as described in U.S. patent application Ser. No. 08/434,465, filed May 4, 1995, entitled “Nucleic Acid Ligand Complexes”.

In certain aspects of the present invention it is desirable to provide a complex comprising one or more nucleic acid ligands to components of the C5 protein covalently linked with a non-immunogenic, high molecular weight compound or lipophilic compound. A non-immunogenic, high molecular weight compound is a compound between approximately 100 Da to 1,000,000 Da, more preferably approximately 1000 Da to 500,000 Da, and most preferably approximately 1000 Da to 200,000 Da, that typically does not generate an immunogenic response. For the purposes of this invention, an immunogenic response is one that causes the organism to make antibody proteins. In one preferred embodiment of the invention, the non-immunogenic, high molecular weight compound is a polyalkylene glycol. In the most preferred embodiment, the polyalkylene glycol is polyethylene glycol (PEG). More preferably, the PEG has a molecular weight of about 10-80K. Most preferably, the PEG has a molecular weight of about 20-45K. In certain embodiments of the invention, the non-immunogenic, high molecular weight compound can also be a nucleic acid ligand.

### Antibodies

In a specific feature, compounds of the present invention are useful to identify binding molecules which inhibit complement pathway functions

Compounds of the present invention (i.e., epitopes of C5), including portions or fragments thereof, can be used as immunogens to generate binding molecules, preferably antibodies, that bind to C5 polypeptides using standard techniques for polyclonal and monoclonal antibody preparation. The compounds of the present invention comprise at least 4 amino acid residues of the amino acid sequence shown in SEQ ID NOs: 1, 3, and 5 and encompass linear and non-linear epitopes such that a binding molecule which binds to antigenic portions of a C5 peptide in such a way as to form a specific immune complex. Preferably, compounds comprise at least 6, 8, 10, 15, 20, or 30 amino acid residues. Longer peptides are sometimes preferable over shorter peptides, depending on use and according to methods well known to someone skilled in the art.

Typically, a peptide is used to prepare antibodies by immunizing a suitable subject, (e.g., rabbit, goat, mouse, or other mammal) with the immunogen. An appropriate immunogenic preparation can contain, for example, a recombinant alternative pathway component, e.g., C5 protein, or a portion or fragment thereof, or a chemically synthesized alternative pathway component, e.g., C5 peptide or antagonist. See, e.g., U.S. Pat. Nos. 5,460,959, 5,601,826, 5,994,127, 6,048,729, 6,083,725, each of which is hereby expressly incorporated by reference in their entirety. The preparation can further include an adjuvant, such as Freund's complete or incomplete adjuvant, or similar immunostimulatory agent. Immunization of a suitable subject with an immunogenic alternative pathway component, e.g., C5, or a portion or fragment thereof induces a polyclonal antibody response.

For each of the independently proposed epitope sequences, SEQ ID No 1, 3, 5 an antibody or antibodies binding to all of the amino acid residues identified, or portions of the amino acid residues identified, as a part of an antibody recognition site, would be expected to be in close proximity of the cleavage site on the alpha chain and/or beta chain of C5, which is proteolyzed by the C5 convertases of the alternative or classical pathways. Therefore it is proposed that by binding to epitopes within the proximity of the C5 alpha or beta cleavage site, such antibodies would have the potential to inhibit the cleavage of C5 by functionally inhibiting proteolysis of the cleavage site through steric hindrance.

Binding molecules which bind to or otherwise block the generation and/or activity of the human complement components are envisioned. Thus, binding molecules are useful herein to prevent or inhibit production of C5a and/or the assembly of the membrane attack complex (MAC) associated with C5b. Some binding molecules of the invention include those that associate with complement component C5 thus inhibiting its conversion to C5a and Cb5 leading to assembly of the MAC complex.

A binding molecule “which binds” an antigen of interest, e.g. a C5 polypeptide antigen, is one that binds the antigen with sufficient affinity such that the binding molecule is useful as a diagnostic and/or therapeutic agent in targeting a cell or tissue expressing the antigen, and does not significantly cross-react with other proteins. In one aspect, the extent of binding, e.g., of an antibody to a “non-target” protein will be less than about 10% of the binding of the antibody to its particular target protein as determined by fluorescence activated cell sorting (FACS) analysis or radioimmunoprecipitation (RIA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁶ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁹ M, alternatively at least about 10⁻¹¹ M, alternatively at least about 10⁻¹² M, or greater. In one aspect, the term “specific binding” refers to binding where a compound binds to a particular polypeptide or epitope on a particular polypeptide without substantially binding to any other polypeptide or polypeptide epitope.

Particularly useful binding molecules for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. More particularly, a monoclonal antibody (mAb) may be used. Such an antibody 1) inhibits complement activation in a human body fluid; 2) inhibits the binding of purified human complement component C5 to either human complement component C3 or human complement component C4; and/or 3) does not specifically bind to the human complement activation product for C5a. Particularly useful complement inhibitors are compounds which reduce the generation of C5a and/or C5b-9 by greater than about 30%, 40% or 50% as measured by C5a ELISA or by hemolytic assays.

Functionally, a suitable antibody inhibits the cleavage of C5, which blocks the generation of potent proinflammatory molecules C5a and C5b-9 (terminal complement complex). The preferred anti-C5 antibodies used to treat disorders associated with complement pathway disregulation, preferably ocular diseases in accordance with this disclosure bind to C5 or fragments thereof, e.g., C5a or C5b. Preferably, the anti-C5 antibodies are immunoreactive against epitopes on the alpha and/or beta chain of purified human complement component C5 and are capable of blocking the conversion of C5 into C5a and C5b by C5 convertase. This capability can be measured using the techniques described in Wurzner, et al., Complement Inflamm 8:328-340, 1991.

In a particularly useful aspect, the anti-C5 antibodies are immunoreactive against epitopes on the beta chain, and/or epitopes within the alpha chain of purified human complement component C5, preferably epitopes selected from the group consisting of SEQ ID Nos 1, 3 and 5. In this aspect, the antibodies are also capable of blocking the conversion of C5 into C5a and C5b by C5 convertase. Within the alpha chain, the most preferred antibodies bind to the amino-terminal region, however, they do not bind to free C5a.

Another aspect of the invention is the generation and use of therapeutic antibodies that bind C5 and inhibit its cleavage by only the C5 convertase of the alternative pathway (C3bBbC3b). Such antibodies would be expected to inhibit complement activation resulting from polymorphisms that lead to dysregulation of the alternative pathway without interfering with the normal function of the C5 convertase (C3bC4bC2a) of the classical pathway of complement.

Anti-05 antibodies described herein include human monoclonal antibodies. In some aspects, antigen binding portions of antibodies that bind to C3b, (e.g., V_{H} and V_{L} chains) are “mixed and matched” to create other anti-C5 binding molecules. The binding of such “mixed and matched” antibodies can be tested using the aforementioned binding assays (e.g., ELISAs). When selecting a V_{H} to mix and match with a particular V_{L} sequence, typically one selects a V_{H} that is structurally similar to the V_{H} it replaces in the pairing with that V_{L}. Likewise a full length heavy chain sequence from a particular full length heavy chain/full length light chain pairing is generally replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Identifying structural similarity in this context is a process well known in the art.

In other aspects, the invention provides antibodies that comprise the heavy chain and light chain CDR1s, CDR2s and CDR3s of one or more C5-binding antibodies, in various combinations. Given that each of these antibodies can bind to C5 and that antigen-binding specificity is provided primarily by the CDR1, 2 and 3 regions, the V_{H} CDR1, 2 and 3 sequences and V_{L} CDR1, 2 and 3 sequences can be “mixed and matched” (i.e., CDRs from different antibodies can be mixed and matched). C5 binding of such “mixed and matched” antibodies can be tested using the binding assays described herein (e.g., ELISAs). When V_{H} CDR sequences are mixed and matched, the CDR1, CDR2 and/or CDR3 sequence from a particular V_{H} sequence should be replaced with a structurally similar CDR sequence(s). Likewise, when V_{L} CDR sequences are mixed and matched, the CDR1, CDR2 and/or CDR3 sequence from a particular V_{L} sequence should be replaced with a structurally similar CDR sequence(s). Identifying structural similarity in this context is a process well known in the art.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes as the source of the sequences. In one such system, a human antibody is raised in a transgenic mouse carrying human immunoglobulin genes. The transgenic mouse is immunized with the antigen of interest (e.g., epitopes of C5 and further described below). Alternatively, a human antibody is identified by providing a human immunoglobulin gene library displayed on phage and screening the library with the antigen of interest (e.g., C5 proteins or epitopes).

A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline-encoded sequence, due to, for example, naturally occurring somatic mutations or artificial site-directed mutations. However, a selected human antibody typically has an amino acid sequence at least 90% identical to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

The percent identity between two sequences is a function of the number of identity positions shared by the sequences (i.e., % identity=# of identity positions/total # of positions×100), taking into account the number of gaps, and the length of each gap, that need to be introduced for optimal alignment of the two sequences. The comparison of sequences and determination of percent identity between two sequences is determined using the algorithm of E. Meyers and W. Miller (1988 Comput. Appl. Biosci., 4:11-17) which has been incorporated into the ALIGN program (version 2.0), using a PAM120 weight residue table, a gap length penalty of 12 and a gap penalty of 4.

Typically, a V_{H} or V_{L} of a human antibody derived from a particular human germline sequence will display no more than 10 amino acid differences from the amino acid sequence encoded by the human germline immunoglobulin gene. In certain cases, the V_{H} or V_{L} of the human antibody may display no more than 5, or even no more than 4, 3, 2, or 1 amino acid difference from the amino acid sequence encoded by the germline immunoglobulin gene.

### Camelid Antibodies

Antibody proteins obtained from members of the camel and dromedary (Camelus bactrianus and Calelus dromaderius) family, including New World members such as llama species (Lama paccos, Lama glama and Lama vicugna), have been characterized with respect to size, structural complexity and antigenicity for human subjects. Certain IgG antibodies found in nature in this family of mammals lack light chains, and are thus structurally distinct from the four chain quaternary structure having two heavy and two light chains typical for antibodies from other animals. See WO 94/04678.

A region of the camelid antibody that is the small, single variable domain identified as V_{HH} can be obtained by genetic engineering to yield a small protein having high affinity for a target, resulting in a low molecular weight, antibody-derived protein known as a “camelid nanobody”. See U.S. Pat. No. 5,759,808; see also Stijlemans et al., 2004 J. Biol. Chem. 279: 1256-1261; Dumoulin et al., 2003 Nature 424: 783-788; Pleschberger et al., 2003 Bioconjugate Chem. 14: 440-448; Cortez-Retamozo et al., 2002 Int. J. Cancer 89: 456-62; and Lauwereys. et al., 1998 EMBO J. 17: 3512-3520. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents to detect antigens that are otherwise cryptic using classical immunological techniques, and as possible therapeutic agents. Thus, yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitate drug transport across the blood brain barrier. See U.S. Pat. Pub. No. 20040161738, published Aug. 19, 2004. These features combined with the low antigenicity in humans indicate great therapeutic potential. Further, these molecules can be fully expressed in prokaryotic cells such as E. coli.

Accordingly, a feature of the present invention is a camelid antibody or camelid nanobody having high affinity for C5. In certain aspects herein, the camelid antibody or nanobody is naturally produced in the camelid animal, i.e., is produced by the camelid following immunization with C5 or a peptide fragment thereof, using techniques described herein for other antibodies. Alternatively, an anti-C5 camelid nanobody is engineered, i.e., produced by selection, for example from a library of phage displaying appropriately mutagenized camelid nanobody proteins using panning procedures with C5 or a C5 epitope described herein as a target. Engineered nanobodies can further be customized by genetic engineering to increase the half life in a recipient subject from 45 minutes to two weeks.

### Diabodies

Diabodies are bivalent, bispecific molecules in which V_{H} and V_{L} domains are expressed on a single polypeptide chain, connected by a linker that is too short to allow for pairing between the two domains on the same chain. The V_{H} and V_{L} domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure V_{HA}-V_{LB} and V_{HB}-V_{LA} (V_{H}-V_{L} configuration), or V_{LA}-V_{HB} and V_{LB}-V_{HA} (V_{L}-V_{H} configuration) within the same cell. Most of them can be expressed in soluble form in bacteria.

Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21). A diabody can be fused to Fc to generate a “di-diabody” (see Lu et al., 2004 J. Biol. Chem., 279(4):2856-65).

### Engineered and Modified Antibodies

An antibody of the invention can be prepared using an antibody having one or more V_{H} and/or V_{L} sequences as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain CDRs. For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., 1998 Nature 332:323-327; Jones et al., 1986 Nature 321:522-525; Queen et al., 1989 Proc. Natl. Acad. See. U.S.A. 86:10029-10033; U.S. Pat. No. 5,225,539, and U.S. Pat. Nos. 5,530,101; 5,585,089; 5,693,762 and 6,180,370).

Framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., 1991 Sequences of Proteins of Immunological Interest, Fifth Edition, U.S. Department of Health and Human Services, NIH Publication No. 91-3242; Tomlinson et al., 1992 J. Mol. Biol. 227:776-798; and Cox et al., 1994 Eur. J. Immunol. 24:827-836; the contents of each of which are expressly incorporated herein by reference.

The V_{H} CDR1, 2 and 3 sequences and the V_{L} CDR1, 2 and 3 sequences can be grafted onto framework regions that have the identical sequence as that found in the germline immunoglobulin gene from which the framework sequence is derived, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

CDRs can also be grafted into framework regions of polypeptides other than immunoglobulin domains. Appropriate scaffolds form a conformationally stable framework that displays the grafted residues such that they form a localized surface and bind the target of interest (e.g., C5 antigen). For example, CDRs can be grafted onto a scaffold in which the framework regions are based on fibronectin, ankyrin, lipocalin, neocarzinostain, cytochrome b, CP1 zinc finger, PST1, coiled coil, LAC1-D1, Z domain or tendramisat (See e.g., Nygren and Uhlen, 1997 Current Opinion in Structural Biology, 7, 463-469).

Another type of variable region modification is mutation of amino acid residues within the V_{H} and/or V_{L} CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s), and the effect on antibody binding, or other functional property of interest, can be evaluated in in vitro or in vivo assays as described herein. Conservative modifications can be introduced. The mutations may be amino acid substitutions, additions or deletions. Moreover, typically no more than one, two, three, four or five residues within a CDR region are altered.

Engineered antibodies of the invention include those in which modifications have been made to framework residues within V_{H} and/or V_{L}, e.g., to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed by the invention.

Another type of framework modification involves mutating one or more residues within the framework region, or even within one or more CDR regions, to remove T cell-epitopes to thereby reduce the potential immunogenicity of the antibody. This approach is also referred to as “deimmunization” and is described in further detail in U.S. Pat. Pub. No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another aspect, the antibody is modified to increase its biological half-life. Various approaches are possible. For example, U.S. Pat. No. 6,277,375 describes the following mutations in an IgG that increase its half-life in vivo: T252L, T254S, T256F. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another aspect, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another aspect, one or more amino acid residues are altered to thereby alter the ability of the antibody to fix complement. This approach is described further in WO 94/29351.

In yet another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In still another aspect, the glycosylation of an antibody is modified. For example, an aglycoslated antibody can be made (i.e., the antibody lacks glycosylation). Glycosylation can be altered, for example, to increase the affinity of the antibody for an antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Pub. WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al., 2002 J. Biol. Chem. 277:26733-26740). WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180).

Another modification of the antibodies herein that is contemplated by the invention is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG moieties become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain aspects, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

In addition, pegylation can be achieved in any part of a C5 binding polypeptide of the invention by the introduction of a nonnatural amino acid. Certain nonnatural amino acids can be introduced by the technology described in Deiters et al., J Am Chem Soc 125:11782-11783, 2003; Wang and Schultz, Science 301:964-967, 2003; Wang et al., Science 292:498-500, 2001; Zhang et al., Science 303:371-373, 2004 or in U.S. Pat. No. 7,083,970. Briefly, some of these expression systems involve site-directed mutagenesis to introduce a nonsense codon, such as an amber TAG, into the open reading frame encoding a polypeptide of the invention. Such expression vectors are then introduced into a host that can utilize a tRNA specific for the introduced nonsense codon and charged with the nonnatural amino acid of choice. Particular nonnatural amino acids that are beneficial for purpose of conjugating moieties to the polypeptides of the invention include those with acetylene and azido side chains. The polypeptides containing these novel amino acids can then be pegylated at these chosen sites in the protein.

### Methods of Engineering Antibodies

As discussed above, anti-C5 antibodies can be used to create new anti-C5 antibodies by modifying full length heavy chain and/or light chain sequences, V_{H} and/or V_{L} sequences, or the constant region(s) attached thereto. For example, one or more CDR regions of the antibodies can be combined recombinantly with known framework regions and/or other CDRs to create new, recombinantly-engineered, anti-C5 antibodies. Other types of modifications include those described in the previous section. The starting material for the engineering method is one or more of the V_{H} and/or V_{L} sequences, or one or more CDR regions thereof. To create the engineered antibody, it is not necessary to actually prepare (i.e., express as a protein) an antibody having one or more of the V_{H} and/or V_{L} sequences, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Standard molecular biology techniques can be used to prepare and express the altered antibody sequence. The antibody encoded by the altered antibody sequence(s) is one that retains one, some or all of the functional properties of the anti-C5 antibody from which it is derived, which functional properties include, but are not limited to C5 activities described herein. Functional properties of the altered antibodies can be assessed using standard assays available in the art and/or described herein (e.g., ELISAs).

In certain aspects of the methods of engineering antibodies of the invention, mutations can be introduced randomly or selectively along all or part of an anti-C5 antibody coding sequence and the resulting modified anti-C5 antibodies can be screened for binding activity and/or other functional properties (e.g., inhibiting MAC formation, modulating complement pathway dysregulation) as described herein. Mutational methods have been described in the art. For example, PCT Pub. WO 02/092780 by Short describes methods for creating and screening antibody mutations using saturation mutagenesis, synthetic ligation assembly, or a combination thereof. Alternatively, WO 03/074679 by Lazar et al. describes methods of using computational screening methods to optimize physiochemical properties of antibodies.

A nucleotide sequence is said to be “optimized” if it has been altered to encode an amino acid sequence using codons that are preferred in the production cell or organism, generally a eukaryotic cell, for example, a cell of a yeast such as Pichia, an insect cell, a mammalian cell such as Chinese Hamster Ovary cell (CHO) or a human cell. The optimized nucleotide sequence is engineered to encode an amino acid sequence identical or nearly identical to the amino acid sequence encoded by the original starting nucleotide sequence, which is also known as the “parental” sequence.

### Non-Antibody C5 Binding Molecules

The invention further provides C5 binding molecules that exhibit functional properties of antibodies but derive their framework and antigen binding portions from other polypeptides (e.g., polypeptides other than those encoded by antibody genes or generated by the recombination of antibody genes in vivo). The antigen binding domains (e.g., C5 binding domains or epitopes of the present invention) of these binding molecules are generated through a directed evolution process. See U.S. Pat. No. 7,115,396. Molecules that have an overall fold similar to that of a variable domain of an antibody (an “immunoglobulin-like” fold) are appropriate scaffold proteins. Scaffold proteins suitable for deriving antigen binding molecules include fibronectin or a fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VCAM-1,1-set immunoglobulin domain of myosin-binding protein C, 1-set immunoglobulin domain of myosin-binding protein H, 1-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, and thaumatin.

The antigen binding domain (e.g., the immunoglobulin-like fold) of the non-antibody binding molecule can have a molecular mass less than 10 kD or greater than 7.5 kD (e.g., a molecular mass between 7.5-10 kD). The protein used to derive the antigen binding domain is a naturally occurring mammalian protein (e.g., a human protein), and the antigen binding domain includes up to 50% (e.g., up to 34%, 25%, 20%, or 15%), mutated amino acids as compared to the immunoglobulin-like fold of the protein from which it is derived. The domain having the immunoglobulin-like fold generally consists of 50-150 amino acids (e.g., 40-60 amino acids).

To generate non-antibody binding molecules, a library of clones is created in which sequences in regions of the scaffold protein that form antigen binding surfaces (e.g., regions analogous in position and structure to CDRs of an antibody variable domain immunoglobulin fold) are randomized. Library clones are tested for specific binding to the epitopes of interest (e.g., C5) and for other functions (e.g., inhibition of C5 activity). Selected clones can be used as the basis for further randomization and selection to produce derivatives of higher affinity for the antigen.

High affinity binding molecules are generated, for example, using the tenth module of fibronectin III (¹⁰Fn3) as the scaffold. A library is constructed for each of three CDR-like loops of ¹⁰FN3 at residues 23-29, 52-55, and 78-87. To construct each library, DNA segments encoding sequence overlapping each CDR-like region are randomized by oligonucleotide synthesis. Techniques for producing selectable ¹⁰Fn3 libraries are described in U.S. Pat. Nos. 6,818,418 and 7,115,396; Roberts and Szostak, 1997 Proc. Natl. Acad. Sci. USA 94:12297; U.S. Pat. No. 6,261,804; U.S. Pat. No. 6,258,558; and Szostak et al. WO98/31700.

Non-antibody binding molecules can be produces as dimers or multimers to increase avidity for the target antigen. For example, the antigen binding domain is expressed as a fusion with a constant region (Fc) of an antibody that forms Fc-Fc dimers. See, e.g., U.S. Pat. No. 7,115,396.

### Nucleic Acid Molecules Encoding Antibodies of the Invention

Another aspect of the invention pertains to nucleic acid molecules that encode the C5 binding molecules of the invention. The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an aspect, the nucleic acid is a cDNA molecule. The nucleic acid may be present in a vector such as a phage display vector, or in a recombinant plasmid vector.

Nucleic acids sequences of binding molecules can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to an scFv gene. In these manipulations, a V_{L}- or V_{H}-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as to a Fab light chain gene) by operatively linking the V_{L}-encoding DNA to another DNA molecule encoding the light chain constant region, CL. The sequences of human light chain constant region genes are known in the art (see e.g., Kabat et al., 1991 Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or a lambda constant region.

To create an scFv gene, the V_{H}- and V_{L}-encoding DNA fragments are operatively linked to another fragment encoding a flexible linker, e.g., encoding the amino acid sequence (Gly4-Ser)₃, such that the V_{H} and V_{L} sequences can be expressed as a contiguous single-chain protein, with the V_{L} and V_{H} regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554).

### Monoclonal Antibody Generation

Monoclonal antibodies (mAbs) can be produced by a variety of techniques, including conventional monoclonal antibody methodology e.g., the standard somatic cell hybridization technique of Kohler and Milstein (1975 Nature, 256:495), or using library display methods, such as phage display.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539, and U.S. Pat. Nos. 5,530,101; 5,585,089; 5,693,762 and 6,180,370.

In a certain aspect, the antibodies of the invention are human monoclonal antibodies. Such human monoclonal antibodies directed against C5 epitopes can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous p and K chain loci (see, e.g., Lonberg et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N.Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et at., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Pub. Nos. WO 92103918, WO 93/12227, WO 94/25585, WO 97113852, WO 98/24884 and WO 99/45962, all to Lonberg and Kay; and PCT Pub. No. WO 01/14424 to Korman et al.

In another aspect, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM mice”, are described in detail in WO 02/43478.

Still further, alternative transgenic animal systems expressing human immunoglobulin genes are available in the art and can be used to raise anti-C5 antibodies of the invention. For example, an alternative transgenic system referred to as the Xenomouse® (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-C5 antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise anti-C5 antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. Libraries can be screened for binding to full length C5 antigen or to a particular C5 epitopes of SEQ ID 1, 3, 5.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Generation of Human Monoclonal Antibodies in Human Ig Mice

Purified recombinant human C5 expressed in prokaryotic cells (e.g., E. coli) or eukaryotic cells (e.g., mammalian cells, e.g., HEK293 cells) can be used as the antigen. The protein can be conjugated to a carrier, such as keyhole limpet hemocyanin (KLH).

Fully human monoclonal antibodies to C5 neo-epitopes are prepared using HCo7, HCo12 and HCo17 strains of HuMab transgenic mice and the KM strain of transgenic transchromosomic mice, each of which express human antibody genes. In each of these mouse strains, the endogenous mouse kappa light chain gene can be homozygously disrupted as described in Chen et al., 1993 EMBO J. 12:811-820 and the endogenous mouse heavy chain gene can be homozygously disrupted as described in Example 1 of WO 01109187. Each of these mouse strains carries a human kappa light chain transgene, KCo5, as described in Fishwild et al., 1996 Nature Biotechnology 14:845-851. The HCo7 strain carries the HCo7 human heavy chain transgene as described in U.S. Pat. Nos. 5,545,806; 5,625,825; and 5,545,807. The HCo12 strain carries the HCo12 human heavy chain transgene as described in Example 2 of WO 01/09187. The HCo17 stain carries the HCo17 human heavy chain transgene. The KNM strain contains the SC20 transchromosome as described in WO 02/43478.

To generate fully human monoclonal antibodies to C5 epitopes, HuMab mice and KM mice are immunized with purified recombinant C5, a C5 fragment, or a conjugate thereof (e.g., C5-KLH) as antigen. General immunization schemes for HuMab mice are described in Lonberg, N. et al., 1994 Nature 368(6474): 856-859; Fishwild, D. et al., 1996 Nature Biotechnology 14:845-851 and WO 98/24884. The mice are 6-16 weeks of age upon the first infusion of antigen. A purified recombinant preparation (5-50 μg) of the antigen is used to immunize the HuMab mice and KM mice in the peritoneal cavity, subcutaneously (Sc) or by footpad injection.

Transgenic mice are immunized twice with antigen in complete Freund's adjuvant or Ribi adjuvant either in the peritoneal cavity (IP), subcutaneously (Sc) or by footpad (FP), followed by 3-21 days IP, Sc or FP immunization (up to a total of 11 immunizations) with the antigen in incomplete Freund's or Ribi adjuvant. The immune response is monitored by retroorbital bleeds. The plasma is screened by ELISA, and mice with sufficient titers of anti-C5 human immunogolobulin are used for fusions. Mice are boosted intravenously with antigen 3 and 2 days before sacrifice and removal of the spleen. Typically, 10-35 fusions for each antigen are performed. Several dozen mice are immunized for each antigen. A total of 82 mice of the HCo7, HCo12, HCo17 and KM mice strains are immunized with C5 antigens.

To select HuMab or KM mice producing antibodies that bound C5 epitopes, sera from immunized mice can be tested by ELISA as described by Fishwild, D. et al., 1996. Briefly, microtiter plates are coated with purified recombinant C5 at 1-2 μg/ml in PBS, 50 μl/wells incubated 4° C. overnight then blocked with 200 μl/well of 5% chicken serum in PBS/Tween (0.05%). Dilutions of plasma from C5-immunized mice are added to each well and incubated for 1-2 hours at ambient temperature. The plates are washed with PBS/Tween and then incubated with a goat-anti-human IgG Fc polyclonal antibody conjugated with horseradish peroxidase (HRP) for 1 hour at room temperature. After washing, the plates are developed with ABTS substrate (Sigma, A-1888, 0.22 mg/ml) and analyzed by spectrophotometer at OD 415-495. Splenocytes of mice that developed the highest titers of anti-C5 antibodies are used for fusions. Fusions are performed and hybridoma supernatants are tested for anti-C5 activity by ELISA.

The mouse splenocytes, isolated from the HuMab mice and KM mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the number of SP2/0 nonsecreting mouse myeloma cells (ATCC, CRL 1581) with 50% PEG (Sigma). Cells are plated at approximately 1×10⁵/well in flat bottom microtiter plates, followed by about two weeks of incubation in selective medium containing 10% fetal bovine serum, 10% P388D1(ATCC, CRL TIB-63) conditioned medium, 3-5% Origen® (IGEN) in DMEM (Mediatech, CRL 10013, with high glucose, L-glutamine and sodium pyruvate) plus 5 mM HEPES, 0.055 mM 2-mercaptoethanol, 50 mg/ml gentamycin and 1×HAT (Sigma, CRL P-7185). After 1-2 weeks, cells are cultured in medium in which the HAT is replaced with HT. Individual wells are then screened by ELISA for human anti-C5 monoclonal IgG antibodies. Once extensive hybridoma growth occurred, medium is monitored usually after 10-14 days. The antibody secreting hybridomas are replated, screened again and, if still positive for human IgG, anti-C5 monoclonal antibodies are subcloned at least twice by limiting dilution. The stable subclones are then cultured in vitro to generate small amounts of antibody in tissue culture medium for further characterization.

### Generation of Hybridomas Producing Human Monoclonal Antibodies

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% Origen® (IGEN), 4 mM L-glutamine, 1 mM sodium pyruvate, 5 mM HEPES, 0:055 mM 2-mercaptoethanol, 50 units/ml penicillin, 50 □g/ml streptomycin, 50 □g/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using an extinction coefficient of 1.43. The monoclonal antibodies can be aliquoted and stored at −80° C.

### Generation of Transfectomas Producing Monoclonal Antibodies

Antibodies of the invention also can be produced in a host cell transfectoma using, for example, a combination of recombinant DNA techniques and gene transfection methods as is well known in the art (e.g., Morrison, 1985 Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. 1990 Methods in Enzymology 185, Academic Press, San Diego, Calif.). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe et al., 1988 Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665; and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood, 1985 Immunology Today 6:12-13).

Mammalian host cells for expressing the recombinant antibodies of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in Kaufman and Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

### Bispecific Molecules

In another aspect, the present invention features bispecific molecules comprising a C5 binding molecule (e.g., an anti-C5 antibody, or a fragment thereof), of the invention. A C5 binding molecule of the invention can be derivatized or linked to another functional molecule, e.g., another peptide or protein (e.g., another antibody or ligand for a receptor) to generate a bispecific molecule that binds to at least two different binding sites or target molecules. The C5 binding molecule of the invention may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present invention includes bispecific molecules comprising at least one first binding specificity for C5 epitopes and a second binding specificity for a second target epitope.

In one aspect, the bispecific molecules of the invention comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which is expressly incorporated by reference.

The bispecific molecules of the present invention can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al., 1984 J. Exp. Med. 160:1686; Liu et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly aspect, the hinge region is modified to contain an odd number of sulfhydryl residues, for example one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### Measuring Complement Activation

Various methods can be used to measure presence of complement pathway molecules and activation of the complement system (see, e.g., U.S. Pat. No. 6,087,120; and Newell et al., J Lab Clin Med, 100:437-44, 1982). For example, the complement activity can be monitored by (i) measurement of inhibition of complement-mediated lysis of red blood cells (hemolysis); (ii) measurement of ability to inhibit cleavage of C3 or C5; and (iii) inhibition of alternative pathway mediated hemolysis.

The two most commonly used techniques are hemolytic assays (see, e.g., Baatrup et al., Ann Rheum Dis, 51:892-7, 1992) and immunological assays (see, e.g., Auda et al., Rheumatol Int, 10:185-9, 1990). The hemolytic techniques measure the functional capacity of the entire sequence-either the classical or alternative pathway. Immunological techniques measure the protein concentration of a specific complement component or split product. Other assays that can be employed to detect complement activation or measure activities of complement components in the methods of the present invention include, e.g., T cell proliferation assay (Chain et al., J Immunol Methods, 99:221-8, 1987), and delayed type hypersensitivity (DTH) assay (Forstrom et al., 1983, Nature 303:627-629; Halliday et al., 1982, in Assessment of Immune Status by the Leukocyte Adherence Inhibition Test, Academic, New York pp. 1-26; Koppi et al., 1982, Cell. Immunol. 66:394-406; and U.S. Pat. No. 5,843,449).

In hemolytic techniques, all of the complement components must be present and functional. Therefore hemolytic techniques can screen both functional integrity and deficiencies of the complement system (see, e.g., Dijk et al., J Immunol Methods 36: 29-39, 1980; Minh et al., Clin Lab Haematol. 5:23-34 1983; and Tanaka et al., J Immunol 86: 161-170, 1986). To measure the functional capacity of the classical pathway, sheep red blood cells coated with hemolysin (rabbit IgG to sheep red blood cells) are used as target cells (sensitized cells). These Ag-Ab complexes activate the classical pathway and result in lysis of the target cells when the components are functional and present in adequate concentration. To determine the functional capacity of the alternative pathway, rabbit red blood cells are used as the target cell (see, e.g., U.S. Pat. No. 6,087,120).

The hemolytic complement measurement is applicable to detect deficiencies and functional disorders of complement proteins, e.g., in the blood of a subject, since it is based on the function of complement to induce cell lysis, which requires a complete range of functional complement proteins. The so-called CH50 method, which determines classical pathway activation, and the AP50 method for the alternative pathway have been extended by using specific isolated complement proteins instead of whole serum, while the highly diluted test sample contains the unknown concentration of the limiting complement component. By this method a more detailed measurement of the complement system can be performed, indicating which component is deficient.

Immunologic techniques employ polyclonal or monoclonal antibodies against the different epitopes of the various complement components (e.g., C3, C4 an C5) to detect, e.g., the split products of complement components (see, e.g., Hugli et al., Immunoassays Clinical Laboratory Techniques 443-460, 1980; Gorski et al., J Immunol Meth 47: 61-73, 1981; Linder et al., J Immunol Meth 47: 49-59, 1981; and Burger et al., J Immunol 141: 553-558, 1988). Binding of the antibody with the split product in competition with a known concentration of labeled split product could then be measured. Various assays such as radio-immunoassays, ELISA's, and radial diffusion assays are available to detect complement split products.

The immunologic techniques provide high sensitivity to detect complement activation, since they allow measurement of split-product formation in blood from a test subject and control subjects with or without macular degeneration-related disorders. Accordingly, in some methods of the present invention, diagnosis of a disorder associated with ocular disorders is obtained by measurement of abnormal complement activation through quantification of the soluble split products of complement components (e.g., C3a, C4a, C5a, and the C5b-9 terminal complex) in blood plasma from a test subject. The measurements can be performed as described, e.g., in Chenoweth et al., N Engl J Med 304: 497-502, 1981; and Bhakdi et al., Biochim Biophys Acta 737: 343-372, 1983. Preferably, only the complement activation formed in vivo is measured. This can be accomplished by collecting a biological sample from the subject (e.g., serum) in medium containing inhibitors of the complement system, and subsequently measuring complement activation (e.g., quantification of the split products) in the sample.

In the clinical diagnosis or monitoring of patients with disorders associated with ocular diseases or disorders, the detection of complement proteins in comparison to the levels in a corresponding biological sample from a normal subject is indicative of a patient with disorders associated with macular degeneration

In vivo diagnostic or imaging is described in US2006/0067935. Briefly, these methods generally comprise administering or introducing to a patient a diagnostically effective amount of a C5 binding molecule that is operatively attached to a marker or label that is detectable by non-invasive methods. The antibody-marker conjugate is allowed sufficient time to localize and bind to complement proteins within the eye. The patient is then exposed to a detection device to identify the detectable marker, thus forming an image of the location of the C5 binding molecules in the eye of a patient. The presence of C5 binding molecules or complexes thereof is detected by determining whether an antibody-marker binds to a component of the eye. Detection of an increased level in selected complement proteins or a combination of protein in comparison to a normal individual without AMD disease is indicative of a predisposition for and/or on set of disorders associated with macular degeneration. These aspects of the invention are also preferred for use in eye imaging methods and combined angiogenic diagnostic and treatment methods.

In yet another aspect, in a cell-free assay C5 proteins or epitopes can be contacted with a known binding molecule which binds the C5 protein to form an assay mixture, the assay mixture is then contacted with a test compound or binding molecule, to determine the ability of the test compound or binding molecule to interact with the C5 protein over known compounds

### Transgenic Animals

A transgenic animal can be formed using the compounds or binding molecules of the present invention. In particular, transgenic non-human animals can be formed by insertion of the wild type or mutant nucleic acid molecules into cells of a host animal. The insertion of nucleic acid molecules into host animal cells can occur by a variety of methods including but not limited to transfection, particle bombardment, electroporation, and microinjection. Insertions can be made into germ line, embryonic, or mature adult host animal cells.

For example, in one aspect, a host cell of the invention is a fertilized oocyte or an embryonic stem cell into which C5 protein-coding sequences have been introduced. These host cells can then be used to create non-human transgenic animals in which exogenous C5 nucleic acids sequences have been introduced into their genome or homologous recombinant animals in which endogenous C5 sequences have been altered. Such animals are useful for studying the function and/or activity of C5 protein and for identifying and/or evaluating modulators of the protein's activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc.

A transgene is exogenous DNA that is integrated into the genome of a cell from which a transgenic animal develops and that remains in the genome of the mature animal, thereby directing the expression of an encoded gene product in one or more cell types, e.g. liver, or tissues of the transgenic animal. As used herein, a “homologous recombinant animal” is a non-human animal, preferably a mammal, more preferably a mouse, in which an endogenous C5 protein gene has been altered by homologous recombination between the endogenous gene and an exogenous DNA molecule introduced into a cell of the animal, e.g., an embryonic cell of the animal, prior to development of the animal.

A transgenic animal of the invention can be created by introducing C5 protein-encoding nucleic acid into the male pronuclei of a fertilized oocyte (e.g., by micro-injection, retroviral infection) and allowing the oocyte to develop in a pseudopregnant female foster animal. The C5 protein DNA sequence, e.g., one of SEQ ID NOs: 2, 4 or 5, can be introduced as a transgene into the genome of a non-human animal. Alternatively, a non-human homologue of the C5 protein gene, such as a mouse C5 protein gene, can be isolated based on hybridization to the human gene DNA and used as a transgene. Intronic sequences and polyadenylation signals can also be included in the transgene to increase the efficiency of expression of the transgene. A tissue-specific regulatory sequence(s) can be operably-linked to the C5 protein transgene to direct expression of the protein to particular cells, e.g. liver cells. Methods for generating transgenic animals via embryo manipulation and micro-injection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals.

Clones of the non-human transgenic animals can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Diagnostic Assay

Epitope sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample.

In one aspect, the invention encompasses diagnostic assays for determining C5 protein and/or nucleic acid expression as well as C5 protein function, in the context of a biological sample (e.g., blood, serum, cells, tissue) or from individual is afflicted with a disease or disorder, or is at risk of developing a disorder associated with AMD.

Diagnostic assays, such as competitive assays rely on the ability of a labelled analogue (the “tracer”) to compete with the test sample analyte for a limited number of binding sites on a common binding partner. The binding partner generally is insolubilized before or after the competition and then the tracer and analyte bound to the binding partner are separated from the unbound tracer and analyte. This separation is accomplished by decanting (where the binding partner was preinsolubilized) or by centrifuging (where the binding partner was precipitated after the competitive reaction). The amount of test sample analyte is inversely proportional to the amount of bound tracer as measured by the amount of marker substance. Dose-response curves with known amounts of analyte are prepared and compared with the test results in order to quantitatively determine the amount of analyte present in the test sample. These assays are called ELISA systems when enzymes are used as the detectable markers. In an assay of this form, competitive binding between antibodies and anti-C5 antibodies results in the bound C5 protein, preferably the C5 epitopes of the invention, being a measure of antibodies in the serum sample, most particularly, neutralising antibodies in the serum sample.

A significant advantage of the assay is that measurement is made of neutralising antibodies directly (i.e., those which interfere with binding of C5 protein, specifically, epitopes). Such an assay, particularly in the form of an ELISA test has considerable applications in the clinical environment and in routine blood screening.

The invention also pertains to the field of predictive medicine in which diagnostic assays, prognostic assays, pharmacogenomics, and monitoring clinical trials are used for prognostic (predictive) purposes to thereby treat an individual prophylactically.

The invention also provides for prognostic (or predictive) assays for determining whether an individual is at risk of developing a disorder associated with dysregulation of complement pathway activity. For example, mutations in a C5 gene can be assayed in a biological sample. Such assays can be used for prognostic or predictive purpose to thereby prophylactically treat an individual prior to the onset of a disorder characterized by or associated with C5 protein, nucleic acid expression or activity.

Another aspect of the invention provides methods for determining C5 nucleic acid expression or C5 protein activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs) on the expression or activity of C5 protein in clinical trials.

In addition to the use of C5 nucleic acids and proteins in these methods, anti-C5 binding molecules may be used as described above to treat disorders and diseases which, in accordance with the invention, have been discovered to involve neovascularization, inflammation as described above.

### Pharmaceutical Compositions

The compounds and binding molecules of the invention may be administered in free form or in pharmaceutically acceptable salt forms, carriers, excipients and stabilizers. Such compositions may be prepared in conventional manner and exhibit the same order of activity as the free compounds. (Remington's Pharmaceutical Sciences 16th edition, Osol, A. Ed. [1980]),

Utility of the anti-C5 antibody or anti-C5 antibody fragment, e.g. in the treatment of ophthalmic diseases and disorders involving inflammatory or neovascular event, as hereinabove specified, may be demonstrated in animal test methods as well as in clinic, for example in accordance with the methods hereinafter described.

According to the invention, compounds and binding molecules of the invention may be administered by any conventional route, in particular enterally, e.g. orally, e.g. in the form of tablets or capsules, or parenterally (preferably subcutaneously, intravenously, or intracamerally, intravitreally, or subconjunctivally, or subtenon's), e.g. in the form of injectable solutions or suspensions, topically (preferably in an ophthalmic solution administered to the eye), e.g. in the form of solutions, gels, ointments or creams, or in a nasal, transdermal patch or suppository form.

Pharmaceutical compositions comprising compounds and binding molecules of the invention in free form or in pharmaceutically acceptable salt form in association with at least one pharmaceutical acceptable carrier or diluent may be manufactured in conventional manner by mixing with a pharmaceutically acceptable carrier or diluent. Unit dosage forms for oral administration contain, for example, from about 0.1 mg to about 500 mg of active substance.

Preferably, compounds and binding molecules of the invention such as a anti-C5 antibody or fragment thereof are administered topically, e.g. to the surface of the eye, or parenterally, e.g., intravenously, intravitreally, intracamerally, subconjunctivally or subtenon's, or subcutaneously.

Daily dosages required in practicing the method of the present invention will vary depending upon, for example, the compound or binding molecule used, the host, the mode of administration, the severity of the condition to be treated.

Compounds or binding molecules identified by the screening assays disclosed herein can be formulated in an analogous manner, using standard techniques well known in the art

### Articles of Manufacture

In another feature of the invention, an article of manufacture containing materials (e.g., comprising compounds or binding molecules of the present invention) useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide or an antibody of the invention. An instruction or label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The invention having been fully described, is further illustrated by the following examples and claims, which are illustrative and are not meant to be further limiting. Those skilled in the art will recognize or be able to ascertain using no more than routine experimentation, numerous equivalents to the specific procedures described herein. Such equivalents are within the scope of the present invention and claims. The contents of all references, including issued patents and published patent applications, cited throughout this application are hereby incorporated by reference.

### EXAMPLES

### Example 1

In most cases, due to the low conservation of C5 protein sequence between mouse and human, antibodies raised against human C5 do not show binding to mouse C5. Thus, chimeric C5 proteins (containing human and mouse protein sequences) which retain activity in functional assays can be used to determine the epitope of an anti-human anti-C5 antibody.

DNA constructs expressing: human alpha chain/mouse beta chain; or mouse alpha/human beta chain can be used to map antibodies to epitopes on the human alpha or beta chain. DNA for chimeric human/mouse C5 constructs in plasmid form is obtained from GeneArt. Epitopes within a chain are finely mapped using chimeric constructs expressing stretches of 100 amino acid mouse protein sequences grafted into human C5 protein sequences to substitute their respective human sequences. Each chimeric protein contains a stretch of histidines at its C-terminus for affinity purification.

Inserts encoding the chimeric proteins from plasmids are isolated, cloned into mammalian expression vectors (e.g. pCDNA3.1) using standard techniques (see Sambrook, Maniatis, etc.) to produce the encoded protein. Briefly, 293T cells are plated at 6×106 cells/100 mm plate in DMEM (Gibco 11995-073), 10% FBS (Hyclone SH30070.03) without Penicillin-Streptomycin Subsequently, transfection is achieved using 10 μg of plasmid construct containing chimeric human/mouse protein encoding sequences mixed in 750 μl of OPTI-MEM (Gibco 51985-034) final. Transfection mixes are set for ten 100 mm plates. 30 μl Lipofectamine 2000 (Invitrogen 11668-019) is mixed with 720 μl OPTI-MEM (per 100 mm plate). 24 hours after transfection, plates are washed with IS GRO medium (Irvine Scientific 91103), 6 ml of new IS GRO medium is added to each plate and incubated for 24-48 hours. The resulting supernatant is harvested. New IS GRO medium is added to each plate and cells are incubated for 24-48 hours for another harvest. Often, the same process is performed to harvest supernatants a third time.

The supernatant is filtered through a 0.2 micron filter and further purified (alternatively the supernatant may be stored at 80° C. until purification). Conventional purification processes may be used. Briefly, EDTA-free protease inhibitor cocktail tablets (Roche) are added to the supernatant and the pH is adjusted to 8 with NaOH. Ni-NTA resin is equilibrated with PBS, 10 mM imidazole, pH 7.4 and protease inhibitors. Supernatant is bound to the resin (1.5 mL BV) for 1 hour and applied to a gravity flow column. The column is washed and protein is eluted with a solution of PBS, 300 mM imidazole, pH 7.4 and protease inhibitors. Fractions are tested on an electrophoretic gel. The fractions are pooled and dialyzed in PBS pH 7.4 to remove imidazole. The proteins are tested for purity and activity.

### Identification of C5 Antigenic Epitopes

Epitope mapping of anti-C5 antibody fragments (Fabs) and full length IgGs are investigated by using competitive ELISA assays. Competition against 5G1.1 (an alpha chain binder, Thomas T C et al, Molecular Immunology, 33, 1389-1401, (1996)) antibodies and N19/8 (a beta chain binder, Evans M J et al, Molecular Immunology, 332 1183-1195, (1995)) antibodies are also investigated. Several ELISA assays can be used as described further below. One assay uses 5G1.1 or N19/8 coated on a plate use native C5, and detect binding of the antibodies. Chimeric C5 protein is used for which the beta chain of C5 is of mouse origin and the alpha chain is human origin or other chimeric C5 proteins as described above. Another assay involves solution phase competition wherein the Fab or IgG is pre-incubated in at least 10-fold molar excess with biotin-05, then added to a plate coated with anti-C5 Fabs or antibodies and detected with streptavidin-HRP. Results from these experiments indicate if the antibody candidate competes for the same binding site as 5G1.1, N19/8 or other antibody candidates selected. The results also indicate if the test antibody binds the alpha or beta chain a human C5 protein.

5G1.1 Competition with Chimeric Human/Mouse C5

Maxisorp Plate Nunc. 442-404 are coated with anti-human C5 purified Fabs and Mabs at 4 ug/ml in carbonate buffer (Pierce 28382) pH 9.6 in 1000/well volume. Plates are sealed and put at 4 C overnight. Plates are then aspirated and washed three times with PBS/0.5% Tween20 (PBST) 300 μl/well volumes. Plates are blocked with 300 μl/well Syn Block (AbD Serotec BUF034C) and incubated for two hours at room temperature, then washed one time with PBST 300 μl/well volume. Supernatant from transected 293T cells with the mouse/human chimeric C5 protein are diluted 1:8 in diluent (2% BSA Fraction V (Fisher ICN16006980), 0.1% Tween20 (Sigma P1379), 0.1% Triton-x-100 (Sigma P234729), PBS) and 100 μl/well is added, or purified human C5 (Quidel A403) is diluted in diluent to 1 ug/ml and 100 ul/well and added to the plate. The plates are incubated at room temperature for one hour and washed three times with PBST 300 μl/well volume. 5G1.1 IgG is diluted in diluent at 1 μg/ml, and added to the plate 100 μl/well. Plates are incubated at room temperature for one hour and washed three times in PBST. Detection antibody anti-human IgG Fc-HRP (Pierce 31125) is diluted 1:5000 and 100 μl/well is added to the plate. Plates are incubated at room temperature for one hour and washed four times with PBST. TMB substrate (Pierce 34028) is then added at 100 μl/well. Plates are incubated at room temperature for 10 minutes+/−2 minutes and stop solution (2NH2SO4) is added at 50 μl/well. The absorbance is read in Spectramax 450 nm-570 nm.

Competition with Biotinylated Human C5

Maxisorp Plate Nunc. 442-404 are coated with purified anti-human C5 Fabs and IgG at 5 μg/ml in carbonate buffer (Pierce 28382) pH 9.6 in 50 μl/well volume. Plates are sealed and put at room temperature on shaker for 4 hours. Plates are then aspirated and washed three times with PBST. Plates are blocked with 300 μl/well SuperBlock PBS (Pierce 37515) and incubated for two hours at room temperature. Plates are then washed one time with PBST. Anti-human C5 Fab/Mab are diluted in Superblock to a concentration of 5 ug/ml with biotinylated C5 (Morphosys) at a concentration of 0.25 ug/ml and incubated for one hour before adding 50 μl/well to plate. Plates are incubated at room temperature for one hour and washed three times with PBST 300 μl/well volume. Poly-streptavidin-HRP (Endogen N200) is diluted in Superblock 1:5000 and added to the plate 100 μl/well. Plates are incubated at room temperature for 30 minutes and washed three times with PBST. TMB substrate (Pierce 34028) is then added at 100 μl/well. Plates are incubated at room temperature for 10 minutes+/−2 minutes and stop solution (2NH2504) is added at 50 μl/well. The absorbance is read in Spectramax 450 nm-570 nm.

Competitive Assay with 5G1.1 and N19/9

Maxisorp Plate Nunc. 442-404 are coated with anti-human C5 IgG 5G1.1 or N19/8 at 5 ug/ml in carbonate buffer (Pierce 28382) pH 9.6 in 100 μl/well volume. Plates are sealed and put at 4° C. overnight. Plates are then aspirated and washed three times with PBST. Plates are blocked with 300 μl/well diluent/Block (4% BSA Fraction V (Sigma A403), 0.1% Tween20 (Sigma P1379), 0.1% Triton-x-100 (Sigma P234729), PBS) and incubated for two hours at room temperature. Plates are then washed one time with PBST. Anti-human C5 Fab/Mab are diluted in diluent to a concentration of 2.5 μg/ml with purified C5 (Quidel A403) at concentration of 0.5 ug/ml and incubated for 30 minutes before adding 100 μl/well to plate. Plates are incubated at room temperature for one hour. Plates are washed three times with PBST. Anti-his (Roche 11965085001) is diluted in diluent at 200 mU/ml or Goat anti-mouse Ig-HRP (BD Pharmingen 554002) is diluted 1:5000, and added to the plate 100 μl/well. Plates are incubated at room temperature for one hour and washed three times in PBST. TMB substrate (Pierce 34028) is then added 100 μl/well. Plates are incubated at room temperature for 5-10 minutes and stop solution (2NH2SO4) is added 50 μl/well. The absorbance is read in Spectramax 450 nm-570 nm.

### Example 2

### Generation of Human Antibodies by Phage Display

For the generation of antibodies against C5, selections with the MorphoSys HuCAL GOLD® phage display library are carried out. HuCAL GOLD is a Fab library based on the HuCAL concept in which all six CDRs are diversified, and which employs the CYSDISPLAY technology for linking Fab fragments to the phage surface (Knappik et al., 2000 J. Mol. Biol. 296:57-86; Krebs et al., 2001 J. Immunol. Methods 254:67-84; Rauchenberger et al., 2003 J Biol. Chem. 278(40):38194-38205; WO 01/05950, Lohning, 2001).

### Phagemid Rescue, Phage Amplification, and Purification

The HuCAL GOLD library is amplified in 2×YT medium containing 34 μg/ml chloramphenicol and 1% glucose (2×YT-CG). After infection with VCSM13 helper phages at an OD₆₀₀ₙₘ of 0.5 (30 min at 37° C. without shaking; 30 min at 37° C. shaking at 250 rpm), cells are spun down (4120 g; 5 min; 4° C.), resuspended in 2×YT/34 μg/ml chloramphenicol/50 μg/ml kanamycin/0.25 mM IPTG and grown overnight at 22° C. Phages are PEG-precipitated twice from the supernatant, resuspended in PBS/20% glycerol and stored at −80° C.

Phage amplification between two panning rounds is conducted as follows: mid-log phase E. coli TG1 cells are infected with eluted phages and plated onto LB-agar supplemented with 1% of glucose and 34 μg/ml of chloramphenicol (LB-CG plates). After overnight incubation at 30° C., the TG1 colonies are scraped off the agar plates and used to inoculate 2×YT-CG until an OD₆₀₀ₙₘ of 0.5 is reached and VCSM13 helper phages added for infection as described above.

Pannings with HuCAL GOLD

For the selection of antibodies recognizing C5 two different panning strategies are applied. In summary, HuCAL GOLD phage-antibodies are divided into four pools comprising different combinations of VH master genes (pool 1: VH1/5 AK, pool 2: VH3λκ, pool 3: VH2/4/6λκ, pool 4: VH1-6λκ). These pools are individually subjected to three rounds of solid phase panning on human C5 directly coated to Maxisorp plates and in addition three of solution pannings on biotinylated C5 antigen.

The first panning variant is solid phase panning against C5: 2 wells on a Maxisorp plate (F96 Nunc-Immunoplate) are coated with 300 μl of 5 μg/ml C5-each o/n at 4° C. The coated wells are washed 2× with 350 μl PBS and blocked with 350 μl 5% MPBS for 2 h at RT on a microtiter plate shaker. For each panning about 10¹³ HuCAL GOLD phage-antibodies are blocked with equal volume of PBST/5% MP for 2 h at room temperature. The coated wells are washed 2× with 350 μl PBS after the blocking. 300 μl of pre-blocked HuCAL GOLD® phage-antibodies are added to each coated well and incubated for 2 h at RT on a shaker. Washing is performed by adding five times 350 μl PBS/0.05% Tween, followed by washing another four times with PBS. Elution of phage from the plate is performed with 300 μl 20 mM DTT in 10 mM Tris/HCl pH8 per well for 10 min. The DTT phage eluate is added to 14 ml of E. coli TG1, which are grown to an OD₆₀₀ of 0.6-0.8 at 37° C. in 2YT medium and incubated in 50 ml plastic tubes for 45 min at 37° C. without shaking for phage infection. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates and phages were rescued and amplified as described above. The second and third rounds of the solid phase panning on directly coated C5 antigen is performed according to the protocol of the first round, but with increased stringency in the washing procedure.

The second panning variant is solution panning against biotinylated human C5 antigen: For the solution panning, using biotinylated C antigen coupled to Dynabeads M-280 (Dynal), the following protocol is applied: 1.5 ml Eppendorf tubes are blocked with 1.5 ml 2× Chemiblocker diluted 1:1 with PBS over night at 4° C. 200 μl streptavidin coated magnetic Dynabeads M-280 (Dynal) are washed 1× with 200 μl PBS and resuspended in 200 μl 1× Chemiblocker (diluted in 1×PBS). Blocking of beads is performed in pre-blocked tubes over night at 4° C. Phages diluted in 500 μl PBS for each panning condition are mixed with 500 μl 2× Chemiblocker/0.1% Tween 1 h at RT (rotator). Pre-adsorption of phages is performed twice: 50 μl of blocked Streptavidin magnetic beads are added to the blocked phages and incubated for 30 min at RT on a rotator. After separation of beads via a magnetic device (Dynal MPC-E) the phage supernatant (−1 ml) is transferred to a new blocked tube and pre-adsorption was repeated on 50 μl blocked beads for 30 min. Then, 200 nM biotinylated C5 is added to blocked phages in a new blocked 1.5 ml tube and incubated for 1 h at RT on a rotator. 100 μl of blocked streptavidin magnetic beads is added to each panning phage pool and incubated 10 min at RT on a rotator. Phages bound to biotinylated C5 are immobilized to the magnetic beads and collected with a magnetic particle separator (Dynal MPC-E). Beads are then washed 7× in PBS/0.05% Tween using a rotator, followed by washing another three times with PBS. Elution of phage from the Dynabeads is performed adding 300 μl 20 mM DTT in 10 mM Tris/HCl pH 8 to each tube for 10 min. Dynabeads are removed by the magnetic particle separator and the supernatant is added to 14 ml of an E. coli TG-1 culture grown to OD₆₀₀ₙₘ of 0.6-0.8. Beads are then washed once with 200 μl PBS and together with additionally removed phages the PBS was added to the 14 ml E. coli TG-1 culture. For phage infection, the culture is incubated in 50 ml plastic tubes for 45 min at 37° C. without shaking. After centrifugation for 10 min at 5000 rpm, the bacterial pellets are each resuspended in 500 μl 2×YT medium, plated on 2×YT-CG agar plates and incubated overnight at 30° C. Colonies are then scraped from the plates, and phages are rescued and amplified as described above.

The second and third rounds of the solution panning on biotinylated C5 antigen are performed according to the protocol of the first round, except with increased stringency in the washing procedure.

### Subcloning and Expression of Soluble Fab Fragments

The Fab-encoding inserts of the selected HuCAL GOLD® phagemids are sub-cloned into the expression vector pMORPH®X9_Fab_FH to facilitate rapid and efficient expression of soluble Fabs. For this purpose, the plasmid DNA of the selected clones is digested with XbaI and EcoRI, thereby excising the Fab-encoding insert (ompA-VLCL and phoA-Fd), and cloned into the XbaI/EcoRI-digested expression vector pMORPH® X9_Fab_FH. Fabs expressed from this vector carry two C-terminal tags (FLAG™ and 6×His, respectively) for both, detection and purification.

Microexpression of HuCAL GOLD Fab Antibodies in E. coli

Chloramphenicol-resistant single colonies obtained after subcloning of the selected Fabs into the pMORPH® X9_Fab_FH expression vector are used to inoculate the wells of a sterile 96-well microtiter plate containing 100 μl 2×YT-CG medium per well and grown overnight at 37° C. 5 μl of each E. coli TG-1 culture is transferred to a fresh, sterile 96-well microtiter plate pre-filled with 100 μl 2×YT medium supplemented with 34 μg/ml chloramphenicol and 0.1% glucose per well. The microtiter plates are incubated at 30° C. shaking at 400 rpm on a microplate shaker until the cultures are slightly turbid (˜2-4 hrs) with an OD₆₀₀ₙₘ of ˜0.5.

To these expression plates, 20 μl 2×YT medium supplemented with 34 μg/ml chloramphenicol and 3 mM IPTG (isopropyl-R-D-thiogalactopyranoside) is added per well (end concentration 0.5 mM IPTG), the microtiter plates are sealed with a gas-permeable tape, and the plates are incubated overnight at 30° C. shaking at 400 rpm.

Generation of whole cell lysates (BEL extracts): To each well of the expression plates, 40 μl BEL buffer (2×BBS/EDTA: 24.7 g/l boric acid, 18.7 g NaCl/I, 1.49 g EDTA/I, pH 8.0) is added containing 2.5 mg/ml lysozyme and incubated for 1 h at 22° C. on a microtiter plate shaker (400 rpm). The BEL extracts are used for binding analysis by ELISA or a BioVeris M-Series® 384 analyzer.

### Enzyme Linked Immunosorbent Assay (ELISA) Techniques

5 μg/ml of human recombinant C5 antigen in PBS is coated onto 384 well Maxisorp plates (Nunc-Immunoplate) o/n at 4° C. After coating, the wells are washed once with PBS/0.05% Tween (PBS-T) and 2× with PBS. Then the wells are blocked with PBS-T with 2% BSA for 2 h at RT. In parallel, 15 μl BEL extract and 15 μl PBS-T with 2% BSA are incubated for 2 h at RT. The blocked Maxisorp plated are washed 3× with PBS-T before 10 μl of the blocked BEL extracts are added to the wells and incubated for 1 h at RT. For detection of the primary Fab antibodies, the following secondary antibodies are applied: alkaline phosphatase (AP)-conjugated AffiniPure F(ab′)₂ fragment, goat anti-human, -anti-mouse or -anti-sheep IgG (Jackson Immuno Research). For the detection of AP-conjugates fluorogenic substrates like AttoPhos (Roche) are used according to the instructions by the manufacturer. Between all incubation steps, the wells of the microtiter plate are washed with PBS-T three times and three times after the final incubation with secondary antibody. Fluorescence can be measured in a TECAN Spectrafluor plate reader.

Expression of HuCAL GOLD Fab Antibodies in E. coli and Purification

Expression of Fab fragments encoded by pMORPH®X9_Fab_FH in TG-1 cells is carried out in shaker flask cultures using 750 ml of 2×YT medium supplemented with 34 μg/ml chloramphenicol. Cultures are shaken at 30° C. until the OD₆₀₀ₙₘ reaches 0.5. Expression is induced by addition of 0.75 mM IPTG for 20 h at 30° C. Cells are disrupted using lysozyme and Fab fragments isolated by Ni-NTA chromatography (Qiagen, Hilden, Germany). Protein concentrations can be determined by UV-spectrophotometry (Krebs et al. J Immunol Methods 254, 67-84 (2001).

1. An isolated polynucleotide having at least 95% nucleic acid sequence identity to a nucleic acid sequence selected from the group consisting of SEQ ID Nos. 2, 4, and 6.

2. An isolated polynucleotide comprising a nucleic acid sequence selected from the group consisting of SEQ ID Nos. 2, 4, and 6.

3. A vector comprising the polynucleotide of claim 2 operably linked to a control sequence.

4. A host cell comprising the vector of claim 3.

5. An isolated polypeptide having at least 95% amino acid identity to an amino acid sequence selected from the group consisting SEQ ID Nos 1, 3 and 5.

6. An isolated polypeptide comprising an amino acid sequence selected from the group consisting SEQ ID Nos 1, 3 and 5.

7. A method for producing a C5 protein, said method comprising culturing the host cell of claim 4 under conditions suitable for expression of said polypeptide and recovering said polypeptide from the cell culture.

8. The method of claim 7 wherein said C5 proteins comprise epitopes selected from the group consisting of SEQ ID No. 1, 3 and 5.

9. An isolated C5 binding molecule comprising an antigen binding portion of an antibody that specifically binds to a C5 epitope within or overlapping amino acids selected from the group consisting of SEQ ID Nos 1, 3 and 5.

10. The C5 binding molecule of claim 9, wherein the antigen binding portion is cross reactive with a C5 antigen of a non-human primate.

11. The C5 binding molecule of claim 9, wherein the antigen binding portion is cross reactive with a C5 antigen of a rodent species.

12. The C5 binding molecule of claim 9, wherein the antigen binding portion binds to a linear epitope.

13. The C5 binding molecule of claim 9, wherein the antigen binding portion binds to a non-linear epitope.

14. The C5 binding molecule of claim 9, wherein the antigen binding portion binds to a human C5 antigen with a K_{D} equal to or less than 0.1 nM.

15. The C5 binding molecule of claim 9, wherein the antigen binding portion binds to C5 antigen of a non-human primate with a K_{D} equal to or less than 0.3 nM.

16. The C5 binding molecule of claim 9, wherein the antigen binding portion thereof binds to mouse C5 antigen with a K_{D} equal to or less than 0.5 nM.

17. The C5 binding molecule of any preceding claim, wherein the antigen binding portion is an antigen binding portion of a human antibody.

18. The C5 binding molecule of claim 9, wherein the antibody is a humanized antibody.

19. The C5 binding molecule of claim 9, wherein the antigen binding portion is an antigen binding portion of a monoclonal antibody.

20. The C5 binding molecule of claim 9, wherein the antigen binding portion is an antigen binding portion of a polyclonal antibody.

21. The C5 binding molecule of claim 9, wherein the C5 binding molecule is a chimeric antibody.

22. The C5 binding molecule of claim 9, wherein the C5 binding molecule comprises an Fab fragment, an Fab′ fragment, an F(ab′)₂, or an Fv fragment of the antibody.

23. The C5 binding molecule of claim 9, wherein the C5 binding molecule comprises a single chain Fv.

24. The C5 binding molecule of claim 9, wherein the C5 binding molecule comprises a diabody.

25. The C5 binding molecule of claim 9, wherein the antigen binding portion is derived from an antibody of one of the following isotypes: IgG1, IgG2, IgG3 or IgG4.

26. The C5 binding molecule of claim 9, wherein the antigen binding portion is derived from an antibody of one of the following isotypes: IgG1, IgG2, IgG3 or IgG4 in which the Fc sequence has been altered relative to the normal sequence in order to modulate effector functions or alter binding to Fc receptors.

27. The C5 binding molecule of claim 9, wherein the C5 binding molecule inhibit MAC production in a cell.

28. The C5 binding molecule of claim 9, wherein the C5 binding molecule inhibits C5 binding to a convertase.

29. A method of inhibiting MAC synthesis in a cell, the method comprising contacting a cell with a C5 binding molecule.

30. A method of modulating MAC activity in a subject, the method comprising administering to the subject a C5 binding molecule that modulates cellular activities mediated by the complement system.

31. A method of treating or preventing an ocular disorder in a subject, the method comprising administering to the subject an effective amount of a binding molecule which specifically binds to an epitope selected from SEQ ID Nos. 1, 3 and 5.

32. The method of claim 31, wherein the subject's level of MAC is reduced by at least 5%, relative to the level of MAC in a subject prior to administering the binding molecule.

33. The method of claim 31 wherein the binding molecule is administered intravitreally.

34. The method of claim 31 wherein said ocular disorder is selected from the group consisting of macular degeneration, diabetic ocular diseases and disorders, ocular edema, ischemic retinopathy, anterior ischemic optic neuropathy, optic neuritis, cystoid macular edema, retinal diseases and disorders, pathologic myopia, retinopathy of prematurity, vascularized, rejecting, or otherwise inflamed corneas, keratoconjunctivitis sicca, dry eye, uveitis, scleritis, episcleritis, conjunctivitis, keratitis, orbital cellulitis, ocular myositis, thyroid orbitopathy, lacrimal gland and eyelid inflammation.

35. The method of claim 31 wherein said binding molecule is a monoclonal antibody.

36.-40. (canceled)

41. A kit for detecting the presence of C5 proteins comprising a container containing the antibody of claim 9 and instructions for detecting said proteins bound by said antibody.

42. The kit of claim 41 wherein the antibody further comprises a detectable label.

43. A method of treating or inhibiting an ocular disease or disorder, or delaying their progression; the method comprising administering an effective amount of a protein capable of inhibiting the alternate complement pathway to a subject in need of such treatment.
